(19) United States
(12) Patent Application Publication
(10) Pub. No.: US 2009/0022684 A1
(43) Pub. Date: Jan. 22, 2009

(54) Methods for hematopoietic stimulation

(76) Inventor: Paul Morley, Ottawa (CA)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/800,991

(22) Filed: May 7, 2007

Related U.S. Application Data
(60) Provisional application No. 60/798,972, filed on May 9, 2006.

(51) Int. Cl. A61K 38/18; A61K 38/17; A61K 38/20; A61P 35/02

(52) U.S. Cl. 424/85.2; 514/12; 514/8

(57) ABSTRACT

The present invention is directed to new methods for treating a subject in need of hematopoietic stimulation. The present invention is also directed to new methods of neutrophil stimulation in a subject in need thereof. The present invention is also directed to new methods of platelet stimulation in a subject in need thereof. The methods comprise administering to the subject an effective amount of a peptide optionally in combination of a hematopoietic growth factor, wherein the peptide is:
(i) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1), where Xaa=Lys, Leu, Ile, Nle or Met, preferably Xaa=Lys; or
(ii) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 2), cyclized in the form of a lactam between Glu²² and Lys²⁶,
   Xaa=Leu, Ile, Nle or Met,
   Y=NH₂ or OH.
   Preferably, Xaa is Leu and Y is NH₂.

### RELATED APPLICATION

This application claims the benefit of U.S. application Ser. No. 60/798,972, filed on May 9, 2006, and is incorporated herein by reference in its entirety.

### BACKGROUND OF THE INVENTION

Most of the red blood cells and white blood cells that circulate in the blood have a limited life span. They are constantly formed through the division of pluripotential hematopoietic stem cells (herein referred to as “hematopoietic stem cells”) located in the bone marrow. The process of blood cell formation is called hematopoiesis. Hematopoietic stem cells give rise to hematopoietic progenitor cells, which are cells capable of differentiation into blood cells with a limited lineage.

Hematopoietic stem and progenitor cells have therapeutic potentials due to their ability to restore blood and immune cell functions. Hematopoietic stem cell transplantations, including bone marrow and cord blood transplantations, are medical procedures in the field of hematology and oncology that are currently used to treat a variety of life-threatening diseases, such as leukemia, lymphoma, myeloma and other diseases of the blood or bone marrow.

The ability of the transplanted hematopoietic stem cells to produce blood cells is crucial for the success of hematopoietic stem cell transplantation procedures. It is also very important for the donor of hematopoietic stem cells to be able to restore and maintain the production of their own blood cells after the transplantation. Therefore, methods that can stimulate hematopoesis are highly desirable for hematopoietic stem cell transplantation and other disease treatments that will benefit from thereof.

### SUMMARY OF THE INVENTION

The present invention is based on unexpected discoveries that a Parathyroid Hormone/Parathyroid Hormone-related Protein (herein referred to as “PTH/PTHrP”) agonist described herein optionally in combination with a hematopoietic growth factor can stimulate hematopoiesis, especially neutrophil and platelet production, in a subject. For example, a significant increase in white blood cell population, especially neutrophil population, in healthy adults treated with the cyclic peptide of SEQ ID NO: 4 was observed, compared to those treated with placebo (see Example 4). In addition, mice treated with the cyclic peptide of SEQ ID NO: 4 and granulocyte colony-stimulating factor (herein referred to as “G-CSF”), a hematopoetic growth factor, showed synergistic increase in the white blood cell population compared to mice treated with the peptide or G-CSF alone (see Example 3).

The present invention is directed to a method for treating a subject in need of hematopoetic stimulation by administering an effective amount of a PTH/PTHrP agonist described herein optionally in combination with an effective amount of a hematopoietic growth factor. In one preferred embodiment of the invention, the peptide used in the disclosed method is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1). Xaa is selected from the group consisting of Lys, Leu, Ile, Nle and Met. Preferably, Xaa is Lys (SEQ ID NO: 2). In another preferred embodiment of the invention, the peptide used in the disclosed method is the cyclic peptide Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶. Xaa is selected from the group consisting of Leu, Ile, Nle and Met and Y is NH₂ or OH. Preferably, Xaa is Leu and Y is NH₂ (SEQ ID NO: 4).

The present invention is also directed to the use of a PTH/PTHrP agonist optionally in combination with a hematopoietic growth factor described herein for the manufacture of a medicament for hematopoietic stimulation in a subject in need thereof. The PTH/PTHrP agonist is preferred to be the peptide of SEQ ID NO: 1, SEQ ID NO: 2, SEQ ID NO: 3, or SEQ ID NO: 4.

Another embodiment of the present invention is directed to a method for neutrophil stimulation in a subject in need thereof by administering an effective amount of a PTH/PTHrP agonist described herein optionally in combination with an effective amount of a hematopoietic growth factor. In a preferred embodiment, the peptide used in the disclosed method is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1). Xaa is selected from the group consisting of Lys, Leu, Ile, Nle and Met. Preferably, Xaa is Lys (SEQ ID NO: 2). In another preferred embodiment, the peptide used in the disclosed method is the cyclic peptide Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶. Xaa is selected from the group consisting of Leu, Ile, Nle and Met and Y is NH₂ or OH. Preferably, Xaa is Leu and Y is NH₂ (SEQ ID NO: 4).

The present invention is also directed to the use of a PTH/PTHrP agonist optionally in combination with a hematopoietic growth factor described herein for the manufacture of a medicament for neutrophil stimulation in a subject in need thereof. The PTH/PTHrP agonist is preferred to the peptide of SEQ ID NO: 1, SEQ ID NO: 2, SEQ ID NO: 3, or SEQ ID NO: 4.

Another embodiment of the present invention is directed to a method for platelet stimulation in a subject in need thereof by administering an effective amount of a PTH/PTHrP agonist described herein optionally in combination with an effective amount of a hematopoietic growth factor. In a preferred embodiment, the peptide used in the disclosed method is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1). Xaa is selected from the group consisting of Lys, Leu, Ile, Nle and Met. Preferably, Xaa is Lys (SEQ ID NO: 2). In another preferred embodiment, the peptide used in the disclosed method is the cyclic peptide Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶. Xaa is selected from the group consisting of Leu, Ile, Nle and Met and Y is NH₂ or OH. Preferably, Xaa is Leu and Y is NH₂ (SEQ ID NO: 4).

The present invention is also directed to the use of a PTH/PTHrP agonist optionally in combination with a hematopoietic growth factor described herein for the manufacture of a medicament for platelet stimulation in a subject in need thereof. The PTH/PTHrP agonist is preferred to the peptide of SEQ ID NO: 1, SEQ ID NO: 2, SEQ ID NO: 3, or SEQ ID NO: 4.

The method of the present invention can be used in bone marrow transplantation and cord blood transplantation as well as for the treatment of a variety of diseases or disorders that will benefit from hematopoietic stimulation, neutrophil stimulation and/or platelet stimulation. The method of the present invention can also be used to treat myelosuppression as a consequence of chemotherapy or radiation therapy.

### BRIEF DESCRIPTION OF THE DRAWINGS

FIG. 1 is a plot showing the mean values of white blood cell population for mice in each treatment groups. As a control, mice in treatment group 1 did not receive any treatment. Mice in treatment group 2 were treated with the peptide of SEQ ID NO: 4 only. Mice in treatment group 3 were treated with the peptide of SEQ ID NO: 4, EPO and G-CSF. Mice in treatment group 4 were treated with the peptide of SEQ ID NO: 4 and EPO. Mice in treatment group 5 were treated with the peptide of SEQ ID NO: 4 and G-CSF. Mice in treatment group 6 and 7 were treated with EPO or G-CSF, respectively. Mice in treatment group 8 were treated with EPO and G-CSF.

FIG. 2 is a plot showing the mean values of neutrophil population for mice in each treatment groups. Treatment groups are defined as described for FIG. 1.

FIG. 3 is a plot showing the mean values of lymphocyte population for mice in each treatment groups. Treatment groups are defined as described for FIG. 1.

FIG. 4 is a plot showing the mean plasma concentration of [Leu²⁷]cyclo(Glu²²-Lys²⁶)-hPTH-(1-31 )-NH₂ versus time (linear scale) on Day 1 and Day 7 for subjects in each treatment group. Subjects in treatment group A were treated with 10 μg of the peptide. Subjects in treatment group B were treated with 20 μg of the peptide. Subjects in treatment group C were treated with 40 μg of the peptide. Subjects in treatment group D were treated with 60 μg of the peptide. Subjects in treatment group E were treated with 80 μg of the peptide.

FIG. 5 is a plot showing the mean plasma concentration [Leu 27]cyclo(Glu²²-Lys²⁶)-hPTH-(1-31)-NH₂ versus time (semi-log scale) on Day 1 and Day 7 for subjects in each treatment group. Treatment groups are defined as described in FIG. 3.

FIG. 6 is a plot showing correlation between AUC₍₀₋ₜₗₐₛₜ₎/Dose (semi-log scale) versus Dose (Day 1) and AUC_{(0-τ)}/Dose (semi-log scale) versus Dose (Day 7).

FIG. 7 is a plot showing correlation between Cₘₐₓ/Dose (semi-log scale) versus Dose (Days 1 and 7).

### DETAILED DESCRIPTION OF THE INVENTION

The present invention provides new methods for hematopoietic stimulation by administering to a subject in need thereof an effective amount of a PTH/PTHrP agonist optionally in combination with an effective amount of a hematopoietic growth factor.

As used herein, a “PTH/PTHrP agonist” is a species that can activate the PTH/PTHrP receptor, including PTH(1-84), PTHrP, and fragments, variants and analogs thereof. The activity of PTH/PTHrP agonist can be determined in the ovarectomized rat model of osteoporosis reported by Kimmel et al., Endocrinology, 1993, 32(4):1577. For example, fragments of PTH that can be used in the present invention include PTH(1-31), PTH(1-32), PTH(1-33), PTH(1-34), PTH(1-35), PTH(1- 36), PTH(1-37), PTH(1-38), PTH(1-39), PTH(1-40), PTH(1-41), PTH(28-48), PTH(1-25) variants, PTH(25-39) and C-terminal amide (—C(═O)—NH₂) of any of the foregoing. Suitable PTH/PTHrP analogs for use in accordance with the present invention include those described in U.S. Pat. Nos. 5,589,452, 5,849,695, 5,695,955, 6,362,163, 6,147,186 and 6,583,114 and the teachings of which are hereby incorporated by reference. Cyclic PTH analogs described in U.S. Pat. Nos. 5,556,940, 5,955,425, 6,110,892, 6,316,410 and 6,316,450 can also be used in the present invention and the teachings of which are hereby incorporated by reference.

In one preferred embodiment of the invention, the peptide used for hematopoietic stimulation is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1). Xaa is selected from the group consisting of Lys, Leu, Ile, Nle and Met. Preferably, Xaa is Lys (SEQ ID NO: 2), i.e. the peptide is hPTH-(1-31)-NH₂. In another preferred embodiment of the invention, the peptide used for hematopoieitic stimulation is the cylic peptide Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶, i.e., the carboxylic acid in the side chain of the glutamic acid at position 22 and the amine in the side chain of the lysine at position 26 form a lactam. Xaa is selected from the group consisting of Leu, Ile, Nle and Met and Y is NH₂ or OH. Preferably, Xaa is Leu and Y is NH₂ (SEQ ID NO: 4), i.e., the peptide is [Leu²⁷]cyclo(Glu²²-Lys²⁶)-hPTH-(1-31)-NH₂. In one embodiment of the present invention, the method comprises the step of administering to a subject in need of hematopoietic stimulation an effective amount of a PTH/PTHrP agonist in combination with an effective amount of a hematopoietic growth factor. The PTH/PTHrP agonist is preferred to be the peptide of the peptide of SEQ ID NO: 1, SEQ ID NO: 2, SEQ ID NO: 3, or SEQ ID NO: 4. The hematopoietic growth factor can be administered concurrently with the PTH/PTHrP agonist or non-concurrently. When the hematopoietic growth factor is administered concurrently with the PTH/PTHrP agonist, it can be in the same composition as the PTH/PTHrP agonist or in a different composition from that of the PTH/PTHrP agonist. The administration route and administration schedule for the hematopoietic growth factor can be same as or different from that for the PTH/PTHrP agonist. In another embodiment of the present invention, the method comprises the step of administering to a subject in need of hematopoietic stimulation an effective amount of a PTH/PTHrP agonist in the absence of the hematopoietic growth factor. The PTH/PTHrP agonist is preferred to be the peptide of SEQ ID NO: 1, SEQ ID NO: 2, SEQ ID NO: 3, or SEQ ID NO: 4.

The present invention also provides new methods for neutrophil stimulation in a subject in need thereof by administering to the subject an effective amount a PTH/PTHrP agonist optionally in combination with an effective amount of a hematopoietic growth factor. The PTH/PTHrP agonist is preferred to be the peptide of SEQ ID NO: 1, SEQ ID NO: 2, SEQ ID NO: 3, or SEQ ID NO: 4. In one embodiment of the present invention, the method comprises the step administering to a subject in need of neutrophil stimulation an effective amount of a PTH/PTHrP agonist in the absence of a hematopoietic growth factor. In another embodiment of the present invention, the method comprises the step of administering to a subject in need of neutrophil stimulation an effective amount of a PTH/PTHrP agonist in combination with an effective amount of a hematopoietic growth factor. The PTH/PTHrP agonist is preferred to be the peptide of SEQ ID NO: 1, SEQ ID NO: 2, SEQ ID NO: 3, or SEQ ID NO: 4. The hematopoietic growth factor can be administered concurrently with the PTH/PTHrP agonist or non-concurrently. When the hematopoietic growth factor is administered concurrently with the PTH/PTHrP agonist, it can be in the same composition as the PTH/PTHrP agonist or in a different composition from that of the PTH/PTHrP agonist. The administration route and administration schedule for the hematopoietic growth factor can be same as or different from that for the PTH/PTHrP agonist.

The present invention also provides new methods for platelet stimulation in a subject in need thereof by administering to the subject an effective amount a PTH/PTHrP agonist optionally in combination with an effective amount of a hematopoietic growth factor. The PTH/PTHrP agonist is preferred to be the peptide of SEQ ID NO: 1, SEQ ID NO: 2, SEQ ID NO: 3, or SEQ ID NO: 4. In one embodiment of the present invention, the method comprises the step administering to a subject in need of neutrophil stimulation an effective amount of a PTH/PTHrP agonist in the absence of a hematopoietic growth factor. In another embodiment of the present invention, the method comprises the step of administering to a subject in need of platelet stimulation an effective amount of a PTH/PTHrP agonist in combination with an effective amount of a hematopoietic growth factor. The PTH/PTHrP agonist is preferred to be the peptide of SEQ ID NO: 1, SEQ ID NO: 2, SEQ ID NO: 3, or SEQ ID NO: 4. The hematopoietic growth factor can be administered concurrently with the PTH/PTHrP agonist or non-concurrently. When the hematopoietic growth factor is administered concurrently with the PTH/PTHrP agonist, it can be in the same composition as the PTH/PTHrP agonist or in a different composition from that of the PTH/PTHrP agonist. The administration route and administration schedule for the hematopoietic growth factor can be same as or different from that for the PTH/PTHrP agonist.

As used herein, “hematopoietic stimulation” refers to a treatment which increases the number of blood cells, especially white blood cells, in a subject. The treatment can, for example, promote the survival and growth of implanted hematopoietic stem cells in bone marrow or cord blood transplantation, thereby increasing the efficiency of these transplantation techniques. Alternatively, the treatment can, for example, elevate the number of blood cells especially white blood cells, in subjects with diminished levels of these cells.

As used herein, “neutrophil stimulation” refers to a treatment which increases the number of neutrophils in a subject. The treatment can, for example, elevate the number of neutrophils in subjects with diminished level of neutrophils.

As used herein, “platelet stimulation” refers to a treatment which increases the number of platelets in a subject. The treatment can, for example, elevate the number of platelets in subjects with diminished level of platelets.

As used herein, “hematopoietic growth factor” refers to factor that promotes the survival, proliferation and/or differentiation of hematopoietic stem or progenitor cells to increase the production of blood cells.

In a specific embodiment of the invention, the hematopoietic growth factor is a colony stimulating factor. A “colony stimulating factor” is a substance that promotes the differentiation and proliferation of hematopoietic progenitor cells. More specifically, the colony stimulating factor is selected from the group consisting of granulocyte colony-stimulating factor (herein referred to as “G-CSF”), granulocyte-macrophage colony-stimulating factor, macrophage colony-stimulating factor and multi-colony-stimulating factor. Even more specifically, the hematopoietic growth factor is granulocyte colony-stimulating factor (G-CSF).

In a related embodiment of the invention, the hematopoietic growth factor is an erythropoiesis regulator. More specifically, the erythropoiesis regulator is erythropoietin (herein referred to as “EPO”). An “erythropoiesis regulator” is a substance that can regulate the production of red blood cells. In another related embodiment of the invention, the hematopoietic growth factor is selected from the group consisting of thrombopoietin, Oncostatin M and interlukins.

The hematopoietic growth factors described above are well known to those skilled in the art and most of them are commercially available. They can be obtained by purification, by recombination methodologies or can be derived or synthesized synthetically.

The method of the present invention can be used to treat a subject with a condition that will benefit from hematopoietic stimulation, e.g. a subject that can benefit from an increase in the number of blood cells, such as white blood cells. In a specific embodiment, the method of the present invention can be used to treat a subject with a condition that will benefit from neutrophil stimulation. In another specific embodiment, the method of the present invention can be used to treat a subject with a condition that will benefit from platelet stimulation.

In one embodiment of the invention, the subject in need of hematopoietic stimulation, neutrophil stimulation or platelet stimulation is subjected to hematopoietic stem cell transplantation. More specifically, hematopoietic stem cell transplantation is bone marrow transplantation. The bone marrow transplantation can be autologous (bone marrow cells obtained from the recipient and then re-implanted into the recipient) or alloegeneic (bone marrow cells obtained from a donor different from the recipient). The subject can be a bone marrow donor or a bone marrow recipient. In another embodiment of the invention, hematopoietic stem cell transplantation is cord blood transplantation, where the hematopoietic stem cells are obtained from infant's umbilical cord and placenta after birth.

In one embodiment of the invention, the subject is in need of hematopoietic stimulation, neutrophil stimulation or platelet stimulation as a consequence of disease treatments that can ablate blood cell levels as a side effect. The disease treatments can be chemotherapy, radiation therapy or treatment with bone marrow suppressive drugs.

In another embodiment of the invention, the subject is in need of hematopoietic stimulation, neutrophil stimulation or platelet stimulation as a consequence of a cancer, most commonly a hematological cancer, i.e. a type of cancer that affects blood, bone marrow and lymph nodes. More specifically, the cancer is selected from the group consisting of myeloma, lymphoma and leukemia. Even more specifically, the cancer is acute myeloid leukemia.

One embodiment of the invention is directed to treating a subject with a condition characterized by a diminished level of blood cells or a defect in blood cells. In a specific embodiment of the invention, the condition is myelodysplastic syndrome. In another specific embodiment of the invention, the condition is myelosuppression. Even more specifically, the condition is neutropenia.

In a related embodiment of the invention, the condition is immunodeficiency. The immunodeficiency can result from a variety of disorders, disease infections or conditions, including immunosuppressed conditions due to leukemia or renal failure, autoimmune disorders (e.g. systemic lupus erythematosus, rheumatoid arthritis, auto-immune thyroiditis, scleroderma, and inflammatory bowel disease), viral infections (e.g. human immunodeficiency virus (HIV)), bacterial infections and parasitic infections. Immunodeficiency can also result from congenital diseases or disorders (e.g. hyperimmunoglobulin M syndrome, CD40 ligand deficiency, IL-2 receptor deficiency, γ-chain deficiency, common variable immunodeficiency, Chediak-Higashi syndrome, and Wiskott-Aldrich syndrome) or due to aging.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein, “treating” or “treatment” refers to obtaining desired pharmacological and/or physiological effect. The effect can be prophylactic such as completely or partially preventing one or more symptoms of a disease or condition. The effect can also be therapeutic such as partially or completely alleviating one or more symptoms of a disease or condition.

A variety of administration routes can be used in accordance with the present invention. An effective amount of the peptide described herein and a hematopoietic growth factor can be administered parenterally, orally, by inhalation, topically, rectally, nasally, buccally, vaginally or via an implanted reservoir.

In a preferred embodiment of the invention, an effective amount of the peptide and the hematopoietic growth factor described herein can be administered parenterally. The term “parenteral” as used herein includes subcutaneous, intravenous, intramuscular, intra-articular, intra-synovial, intrasternal, intrathecal, intrahepatic, intralesional and intracranial injection or infusion techniques. More preferably, the route of administration is subcutaneous administration.

Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Composition suitable for oral administration can be in any orally acceptable dosage form including, but not limited to, capsules, tablets, aqueous suspensions or solutions. In the case of tablets for oral use, carriers that are commonly used include lactose and corn starch. Lubricating agents, such as magnesium stearate or sodium stearyl fumarate, are also typically added. For oral administration in a capsule form, useful diluents include lactose and starch (e.g. dried cornstarch or pregelatinized starch). Other useful excipients include colloidal silicon dioxide, microcrystalline cellulose, and sucrose. When aqueous suspensions are required for oral use, the active ingredient is combined with emulsifying and suspending agents. If desired, certain sweetening, flavoring or coloring agents may also added.

Rectal administration includes administering the pharmaceutical compositions into the rectum or large intestine. This can be accomplished using suppositories or enemas. Suppository formulations can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The peptide and the hematopoietic growth factor described herein can also be administered topically. Composition suitable for topical administration can be formulated in a suitable ointment containing the active component suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, composition suitable for topical administration can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Transdermal administration includes percutaneous absorption of the composition through the skin. Transdermal formulations include patches, ointments, creams, gels, salves and the like.

The present invention includes nasally administering to the subject a therapeutically effective amount of the peptide and the hematopoietic growth factor of the present invention. As used herein, nasally administering or nasal administration includes administering the composition to the mucous membranes of the nasal passage or nasal cavity of the patient. As used herein, pharmaceutical compositions for nasal administration of a composition prepared by well-known methods to be administered, for example, as a nasal spray, nasal drop, suspension, gel, ointment, cream or powder. Administration of the composition may also take place using a nasal tampon or nasal sponge.

In addition to the usual meaning of administering the formulations described herein to any part, tissue or organ whose primary function is gas exchange with the external environment, for purposes of the present invention, “pulmonary” is also meant to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

Pharmaceutically acceptable compositions of the PTH/PTHrP agonist and the hematopoietic growth factor described herein can be used according to the method of the present invention. The pharmaceutical compositions described herein can optionally include one or more pharmaceutically acceptable excipients. Such pharmaceutically acceptable excipients are well known in the art and include, for example, salts (such as protamine sulfate, disodium hydrogen phosphate, potassium hydrogen phosphate, sodium chloride, zinc salts, colloidal silica and magnesium trisilicate), surfactant(s), water-soluble polymers (such as polyvinyl pyrrolidone, cellulose based substances, polyethylene glycol, polyacrylates, sodium carboxymethylcellulose, waxes and polyethylene-polyoxypropylene-block polymers), preservatives, antimicrobials, antioxidants, cryo-protectants, wetting agents, viscosity agents, tonicity modifying agents, levigating agents, absorption enhancers, penetration enhancers, pH modifying agents, muco-adhesive agents, coloring agents, flavoring agents, diluting agents, emulsifying agents, suspending agents, solvents, co-solvents, buffers (such as phosphates, glycine, sorbic acid, potassium sorbate and partial glyceride mixtures of saturated vegetable fatty acids), serum proteins (such as human serum albumin), ion exchangers and combinations of these excipients.

The excipient included within the pharmaceutical compositions of the invention is chosen based on the expected route of administration of the composition in therapeutic applications. Accordingly, compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Solid dosage forms, such as tablets, pills and capsules, may also contain one or more binding agents, filling agents, suspending agents, disintegrating agents, lubricants, sweetening agents, flavoring agents, preservatives, buffers, wetting agents, disintegrants, effervescent agents, and other excipients. Such excipients are known in the art. Examples of filling agents are lactose monohydrate, lactose anhydrous, and various starches. Examples of binding agents are various celluloses and cross-linked polyvinylpyrrolidone, microcrystalline cellulose, and silicifized microcrystalline cellulose (SMCC). Suitable lubricants, including agents that act on the flowability of the powder to be compressed, are colloidal silicon dioxide, talc, stearic acid, magnesium stearate, calcium stearate, and silica gel. Examples of sweeteners are any natural or artificial sweetener, such as sucrose, xylitol, sodium saccharin, cyclamate, aspartame, and accsulfame K. Examples of flavoring agents are bubble gum flavor, fruit flavors, and the like. Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride. Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, lactose such as lactose monohydrate, lactose anhydrous, dibasic calcium phosphate, mannitol, starch, sorbitol, sucrose and glucose. Suitable disintegrants include corn starch, potato starch, and modified starches, crosspovidone, sodium starch glycolate, and mixtures thereof. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propyl parabens as preservatives, a dye and a flavoring such as cherry or orange flavor, and the like.

As used herein, “an effective amount” refers to an amount effective to obtain therapeutic or prophylactic effect without including unacceptable side effects. Determination of the effective amount is well within the capability of those skilled in the art, especially in light of the detailed disclosure provided herein. Toxicity and therapeutic efficacy of such compounds can be determined by standard pharmaceutical procedures in cell cultures or experimental animals. The dosage depends upon the dosage form employed and the route of administration utilized. The exact formulation, route of administration, and dosage is chosen by the individual physician in view of the patient's condition. Dosage amount and interval can be adjusted individually to provide plasma levels of the active compound that are sufficient to maintain desired therapeutic effects. In addition to the patient's condition and the mode of administration, the dose administered would depend on the severity of the patient's symptoms and the patient's age and weight. For example, the daily dosage for the peptide of SEQ ID NO: 1, SEQ ID NO: 2 or SEQ ID NO: 3 is preferably in the range of 5 μg to 150 μg. More preferably, the daily dosage is in the range of 30 μg to 100 μg. More preferably, the daily dosage is in the range of 40 μg to 70 μg. Even more preferably, the daily dosage is in the range of 55 μg to 65 μg. Other suitable daily dosage includes 5 μg-25 μg, 25 μg-40 μg, 40 μg-55 μg, 55 μg-65 μg, 65 μg-75 μg, 75 μg-100 μg, 100 μg-125 μg, and 125 μg-150 μg. The daily dosage of the peptide can be administered by any administration route discussed above. More preferably, the daily dosage is administered parenterally. Even more preferably, the daily dosage is administered subcutaneously. In a specific embodiment of the invention, the peptide is administered in an effective amount that results in maximum plasma concentration (herein referred to as “Cₘₐₓ”) of the peptide in the range of 10 to 400 pg/mL. More preferably, the range of Cₘₐₓ is between 20 pg/mL to 300 pg/mL. More preferably, the range of Cₘₐₓ is between 50 pg/mL to 280 pg/mL. More preferably, the range of Cₘₐₓ is between 80 pg/mL to 250 pg/mL. Even more preferably, the range of Cₘₐₓ is between 100 pg/mL to 150 pg/mL. Other suitable dosage range for Cₘₐₓ is 20 pg/mL-40 pg/mL, 40 pg/mL-60 pg/mL, 60 pg/mL-80 pg/mL, 80 pg/mL-100 pg/mL, 100 pg/mL-120 pg/mL, 120 pg/mL-140 pg/mL, 140 pg/mL-160 pg/mL, 160 pg/mL-180 pg/mL, 180 pg/mL-200 pg/mL, 200 pg/mL-230 pg/mL, 230 pg/mL-260 pg/mL, 260 pg/mL-300 pg/mL, 300 pg/mL-350 pg/mL, and 350 pg/mL-400 pg/mL. In another specific embodiment of the invention, the peptide is administered in an effective amount that results in the value for area under the curve (herein referred to as “AUC”) in the plasma peptide concentration versus time cure in the range of 5 μpg·h/mL-400 μpg·h/mL. More preferably, the range of AUC is between 10 pg·h/mL-350 pg·h/mL. More preferably, AUC is in the range of 20 pg·h/mL-300 pg·h/mL. More preferably, AUC is in the range of 50 pg·h/mL-250 μpg·h/mL. More preferably, AUC is in the range of 70 pg·h/mL-200 μpg·h/mL. More preferably, AUC is in the range of 90 μpg·h/mL-150 pg·h/mL. Even more preferably, AUC is in the range of 95 pg·h/mL-125 pg·h/mL. Other suitable range for AUC is 5 μpg·h/mL-20 μpg·h/mL, 20 pg·h/mL-50 pg·h/mL, 50 pg·h/mL-70 pg·h/mL, 70 pg·h/mL-90 pg·h/mL, 90 pg·h/mL-100 μpg·h/mL, 100 pg·h/mL-110 μpg·h/mL, 110 pg·h/mL-120 μpg·h/mL, 120 pg·h/mL-130 pg·h/mL, 130 pg·h/mL-150 pg·h/mL, 150 pg·h/mL-175 pg·h/mL, 175 pg·h/mL-200 μpg·h/mL, 200 pg·h/mL-225 pg·h/mL, 225 pg·h/mL-250 pg·h/mL, 250 pg·h/mL-275 pg·h/mL, 275 pg·h/mL-300 pg·h/mL, 300-350 pg·h/mL, or 350 pg·h/mL-400 pg·h/mL. The peptide can be administered by any administration route discussed above. More preferably, the peptide is administered parenterally. Even more preferably, the peptide is administered subcutaneously. The dosage of other PTH/PTHrP agonists disclosed in the present invention can be adjusted accordingly, depending on the molecular weight and the potency of the specific agonist. An effective amount of the hematopoietic growth factor can be administered by any administration route discussed above. The daily dosage for the hematopoietic growth factor can be determined by those skilled in the art. The dosage depends on the dosage form, administration route and patient's condition. For example, G-CSF can be administered intravenously in the range of 1 mcg/kg to 70 mcg/kg twice daily or subcutaneously in the range of 1 mcg/kg to 3 mcg/kg once daily. G-CSF can also be administered by continuous subcutaneous infusion in the range of 3 mcg/kg/day to 11 mcg/kg/day.

The invention will now be described more specifically with the examples.

### EXAMPLE 1

### Synthesis and Purification Of hPTH-(1-31)-NH₂ (SEQ ID NO: 2)

The peptide hPTH-(1-31)-NH₂ was synthesized and purified as described in U.S. Pat. No. 5,955,425 and the teachings of which is hereby incorporated by reference.

### EXAMPLE 2

### Synthesis and Purification of [Leu²⁷]cyclo(Glu²²-Lys²⁶)-hPTH-(1-31)-NH₂ (SEQ ID NO: 4)

The peptide [Leu²⁷]cyclo(Glu²²-Lys²⁶)-hPTH-(1-31)-NH₂ was synthesized and purified as described in U.S. Pat. No. 5,955,425 and the teachings of which is hereby incorporated by reference.

### EXAMPLE 3

### Synergistic Effect of [Leu²⁷]cyclo(Glu²²-Lys²⁶)-hPTH-(1-31)-NH₂ (SEQ ID NO: 4) and G-CSF in Neutrophil Stimulation in an In Vivo Mouse Study

In vivo Test System. Mouse was selected as the in vivo test system. Sufficient male mice of the C57BL/6 strain was obtained. The animals was obtained as weanlings of about 24-27 days of age on arrival. At randomization, their body weights were within ±20% of the overall mean for each sex. The health care and welfare of the mice were maintained in accordance with the requirement of the Animals (Scientific Procedures) Act 1986 UK. All animals were given clinical inspection for ill health on arrival. They were acclimatized for about three weeks and a veterinary inspection was performed before the start of dosing to ensure their suitability for study. The mice were assigned to treatment groups during the acclimatization period using a total randomization procedure. Group mean body weights were calculated and inspected to ensure there were no unacceptable difference between groups.

Preparation of Administered Formulation of [Leu²⁷]cyclo(Glu²²-Lys²⁶)-hPTH-(1-31)-NH₂. The bulk freeze-dried peptide [Leu²⁷]cyclo(Glu²²-Lys²⁶)-hPTH-(1-31)-NH₂ (SEQ ID NO: 4) was dissolved in 0.01 M acetic acid. The concentration of peptide in the solution was 1 μg/μL. The solution was re-aliquotted into dosing vials and kept frozen at approximately −70° C. The vials were then freeze-dried at the temperature lower than −20° C. and stored frozen until required. The freeze-dried aliquots for injection was reconstituted daily. The peptide was dissolved in an appropriate volume of purified water to an approximate concentration of 2-3 mg/mL. The phosphate-buffered saline (pH=7.4) was added to give the final required concentration and the pH value of the final solution is approximately 7.2. The capped vials were mixed thoroughly to ensure peptide was fully dissolved prior to use. Aseptic techniques and glass vials were used throughout dose preparation.

Preparation of Administered Formulation of G-CSF. G-CSF was provided at a concentration of 300 μg/mL in sorbito, sodium acetate and polysorbate 80 in water for injection. The stock solution was diluted accordingly with water for injection to provide the correct dosing concentration.

Preparation of Administered Formulation of EPO. EPO was provided at a concentration of 40,000 IU/mL in polysorbate 80, sodium chloride, sodium dihydrogen phosphate dihydrate, disodium phosphate dihydrate, glycine and water for injection. The stock solution was diluted accordingly with phosphate buffered solution to provide the correct dosing concentration.

In vivo Mouse Study. The peptide [Leu²⁷]cyclo(Glu²²-Lys²⁶)-hPTH-(1-31 )-NH₂ (SEQ ID NO: 4), G-CSF or EPO were administered subcutaneously over 14 days. The mice were dosed as follows:

The peptide [Leu²⁷]cyclo(Glu²²-Lys^{2 1})-hPTH-(1-3 I)-NH₂ was administered to all mice in Groups 2, 3, 4 and 5 once daily for fourteen days at a dose level of 80 μg/kg.

Mice in Group 3, 4 and 5 also received EPO and/or G-CSF. EPO was administered on Days 1, 4, 7, 10 and 14 at a dose of 600 IU/kg. G-CSF was administered once daily for fourteen days at a dose level of 10 μg/kg.

Mice in 6, 7, 8 received EPO and/or G-CSF. EPO was administered on Days 1, 4, 7, 10 and 14 at a dose level of 600 IU/kg. G-CSF was administered once daily for fourteen days at a dose level of 10 μg/kg.

On Day 14, blood samples (0.5 mL EDTA nominal, 0.6 mL lithium heparin nominal) were withdrawn from all mice and were collected into lithium heparin tubes. The tubes were centrifuged and the plasma were separated and analyzed.

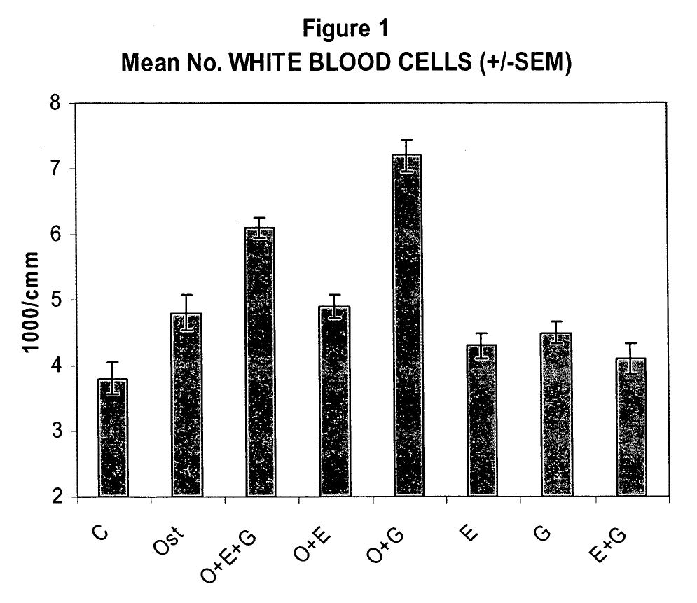

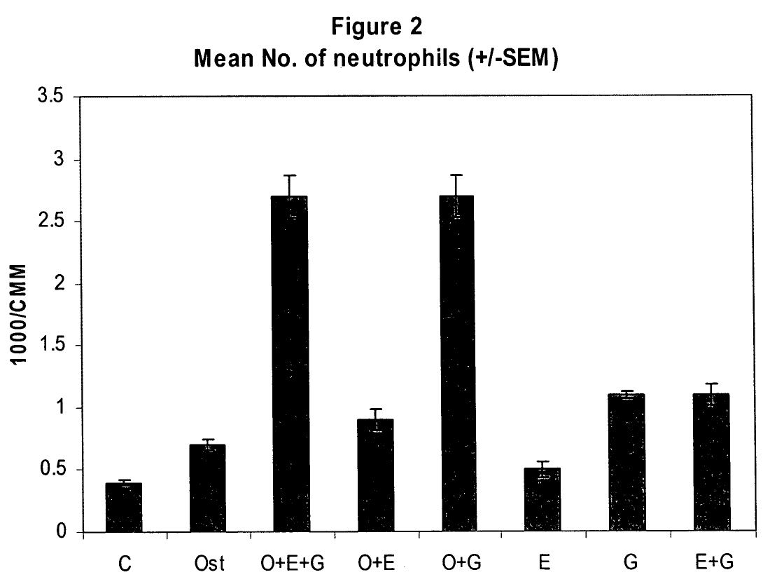

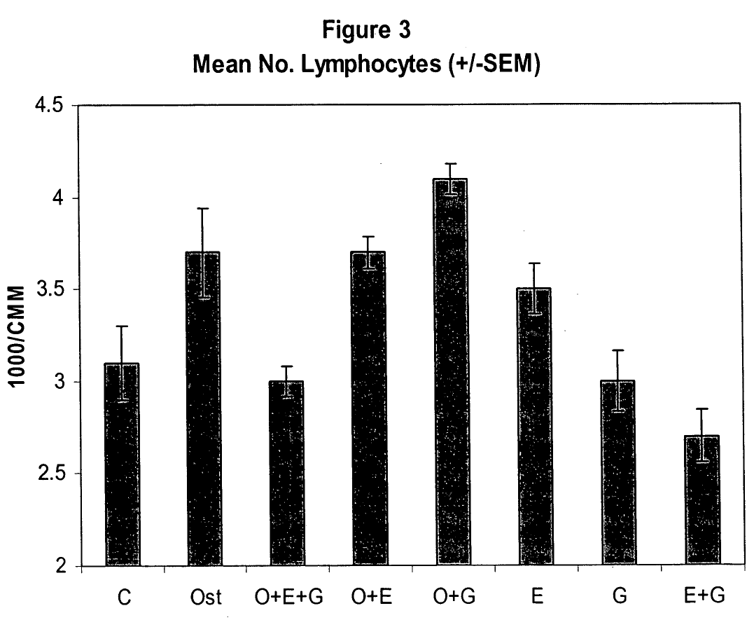

As shown in FIG. 1 and Table 1, mice treated with the peptide alone showed a significant increase (˜26%) in the white blood cell population compared to mice in the control group (without any treatment). Similar increase (˜20%) was observed for mice treated with G-CSF alone. Mice treated with both the peptide and G-CSF showed a surprisingly large increase in white blood cell population compared to mice in the control group. The population of white blood cell almost doubled for mice treated with both the peptide and G-CSF compared to that for mice in the control group. Among white blood cells, neutrophils were especially stimulated. As demonstrated in FIG. 2 and Table 1, the population of neutrophils in mice treated with both the peptide and G-CSF increased almost 6 times compared to that for mice in the control group. Smaller increase was observed for mice treated with the peptide (˜75% increase) or G-CSF (˜175% increase) alone compared to mice in the control group. Moderate increase in lymphocyte population was observed in mice treated with both the peptide and G-CSF (˜30% increase) or with the peptide alone (˜20%), as shown in FIG. 3 and Table 1. These date demonstrated an unexpected synergistic effect of the peptide and G-CSF on the growth of white blood cells, especially neutrophils. The data also demonstrated significant effects of the peptide in stimulating white blood cell population, especially neutrophil population.

TABLE 1
White Blood Cell Populations for Mice in Each Treatment Group

| | | White | Neutro- | Lympho- |
|---|---|---|---|---|
| | | Blood Cells | phils | cytes |
| Group | Treatment | (1000/cmm) | (1000/cmm) | (1000/cmm) |
| 1 | None | 3.8 ± 1.97 | 0.4 ± 0.20 | 3.1 ± 1.61 |
| 2 | Peptide | 4.8 ± 2.13 | 0.7 ± 0.35 | 3.7 ± 1.92 |
| 3 | Peptide + EPO + | 6.1 ± 1.23 | 2.7 ± 1.32 | 3.0 ± 0.68 |
| | G-CSF | | | |
| 4 | Peptide + EPO | 4.9 ± 1.37 | 0.9 ± 0.63 | 3.7 ± 0.72 |
| 5 | Peptide + G-CSF | 7.2 ± 1.96 | 2.7 ± 1.38 | 4.1 ± 0.67 |
| 6 | EPO | 4.3 ± 1.56 | 0.5 ± 0.49 | 3.5 ± 1.12 |
| 7 | G-CSF | 4.5 ± 1.36 | 1.1 ± 0.25 | 3.0 ± 1.30 |
| 8 | EPO + G-CSF | 4.1 ± 1.85 | 1.1 ± 0.68 | 2.7 ± 1.13 |

### EXAMPLE 4

### Effects of [LEU²⁷]CYCLO(GLU²²-LYS²⁶)-hPTH-(1-31)-NH₂ in Neutrophil and Platelet Stimulation in a Phase I Clinical Trial

A Phase I, single-center, randomized double blind, placebo-controlled, multiple-escalating-dose study in healthy older volunteers was carried out for the peptide [Leu²⁷]cyclo(Glu²²-Lys^{2 1})-hPTH-(1-31)-NH₂ (SEQ ID NO: 4). Subjects were healthy males and females (non-reproductive potential), aged 40 to 65 years. Before the study, the volunteers had given informed consent and did not show known sign of osteoporosis or other bone diseases and did not use any medications affecting bone metabolism. Subcutaneous injections of placebo or the peptide with a dosage of 10 μg, 20 μg, 40 μg, 60 μg, or 80 μg was administered daily for 7 days. For each dosage level, 2 subjects (I male and I female) received placebo and 6 subjects (3 males and 3 females) received the peptide daily.

Table 2 shows changes in mean values of selected blood cells from baseline to Day 7 in subject treated with placebo or with the peptide [Leu²⁷]cyclo(G Iu²²-Lys²⁶)-hPTH-(1-31)-NH₂ at each dosage levels. As shown in Table 2, the peptide [Leu²⁷]cyclo(Glu²²-Lys²⁶)-hPTH-(1-31)-NH₂ (SEQ ID NO: 4) has significant stimulating effect on white blood cell populations, especially the neutrophil population. The number of white blood cells and the number neutrophils in a subject treated with the peptide increase as the dosage increases until it reaches the maximum at the dosage level of 60 μg. Increase in the number of white blood cells in subject treated with 60 μg of [Leu²⁷]cyclo(Glu²²-Lys²⁶)-hPTH-(1-31)-NH₂ at Day 7 from the baseline level was about 8 times of that in subjects treated with placebo. More significantly, the increase in population of neutrophils in subjects treated with the peptide at Day 7 was about 50 fold of that for subjects treated with placebo. Significant increase was also observed in platelet count in subjects treated with the peptide at the dosage level of 60 μg and 80 μg.

TABLE 2
Changes in Means for Selected Blood Cell
Populations from Baseline to Day 7

| | White | | Relative | |
|---|---|---|---|---|
| | Blood | | Neutrophil | |
| | Cells | Neutrophil | Count | Platelet |
| Treatment | (x e⁹/L) | (x e⁹/L) | (%) | (x e⁹/L) |
| placebo | 0.59 | 0.09 | 3 | 23.60 |
| 10 μg | 1.50 | 1.22 | 4 | 19.80 |
| 20 μg | 1.75 | 1.22 | 3 | 16.83 |
| 40 μg | 3.23 | 2.88 | 12 | 28.50 |
| 60 μg | 4.65 | 4.53 | 16 | 47.83 |
| 80 μg | 4.40 | 4.40 | 17 | 39.50 |

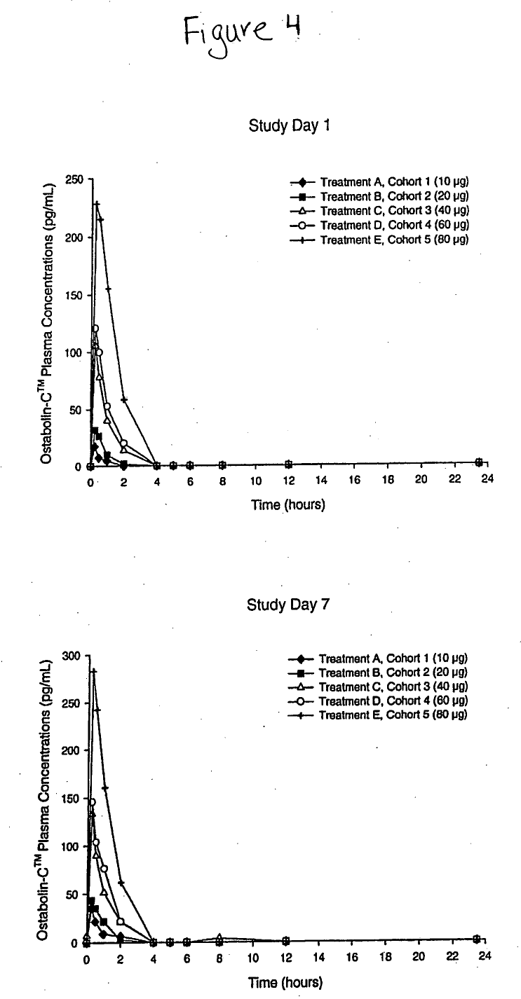

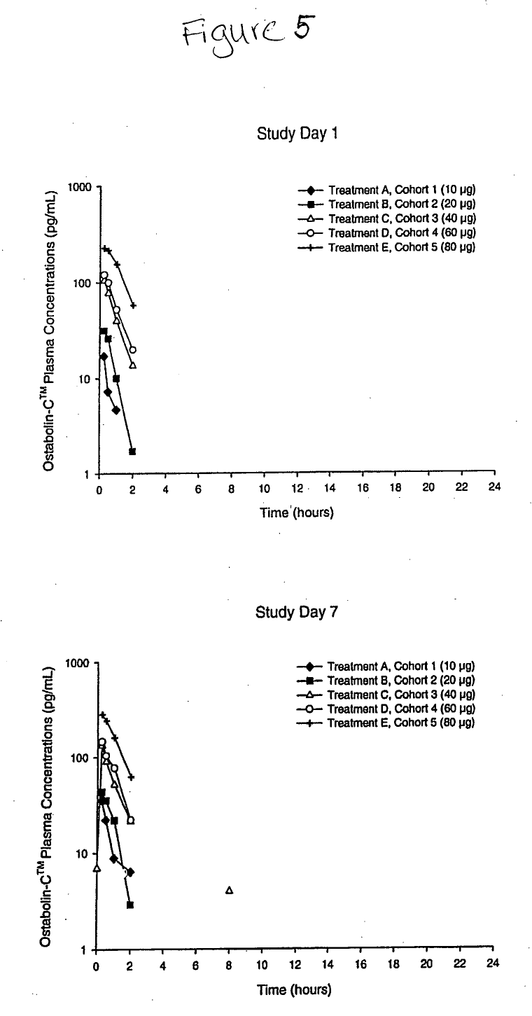

Pharmacokinetics. Plasma concentrations of the peptide were analyzed using the WinNonlin 4.0 Noncompartmental module (Pharsight Inc., Mountainview, Calif.) or equivalent. The following pharmacokinetic parameters were computed from the peptide plasma concentration-time data (See FIGS. 4 and 5):
AUC₍₀₋ₜₗₐₛₜ₎: The area under the plasma concentration versus time curve, from time 0 to the last measurable concentration, as calculated by the linear trapezoidal method (Day 1).
AUC_{(0-τ)}: The area under the plasma concentration versus time curve, from time 0 to the last measurable concentration, as calculated by the linear trapezoidal method (Day 7).
AUC_{(0-∞)} or AUC_{inf}: The area under the plasma concentration versus time curve from time 0 to infinity. AUC_{(0-∞)} is calculated as the sum of the AUC₍₀₋ₜₗₐₛₜ₎ plus the ratio of the last measurable plasma concentration to the elimination rate constant (Cₗₐₛₜ/kel), where Cₗₐₛₜ is the predicted concentration at the time of the last measurable concentration, as determined by linear regression, and kel is the apparent first-order elimination rate constant (Day 1).
AUC/AUC_{inf}: The ratio of AUC_{(0-tlast) to AUC}_{(0-∞)}.
Cₘₐₓ: Maximum measured plasma concentration over the time span specified.
Tₘₐₓ: Time of the maximum measured plasma concentration. If the maximum value occurs at more than one time point, Tₘₐₓ is defined as the first time point with this value.
kel: Apparent first-order terminal elimination rate constant calculated from a semi-log plot of the plasma concentration versus time curve. This parameter was calculated by linear least squares regression analysis using the maximum number of points in the terminal log-linear phase (e.g. three or more non-zero plasma concentrations).
t_{1/2}: The apparent first-order terminal elimination half-life was calculated as 0.693/kel.
Vz/F: Apparent volume of distribution of a drug following subcutaneous administration, calculated as Dose/( AUC_{(0-∞)}·kel), (Day 1).
CL/F: Apparent total body clearance of a drug following oral administration, calculated as Dose/AUC_{(0-∞)}, (Day 1).

Accumulation Index: Calculated as AUC_{(0-τ)} Day 7/AUC(0-tlast) Day 1.

### Pharmacokinetic Parameters

The pharmacokinetic parameters for [Leu²⁷]cyclo(Glu²²-Lys²⁶)-hPTH-(1-31)-NH₂ are summarized by treatment group at each dosage level in Tables 3 and 4.

TABLE 3
Pharmacokinetic results of [Leu²⁷]cyclo(Glu²²-Lys²⁶)-hPTH-(1-31)-
NH₂ per dose level in plasma on Day 1. Geometric mean (CV %) was used
to present the AUC₍₀₋ₜₗₐₛₜ₎, AUC_{(0-∞)}, and Cₘₐₓ pharmacokinetic
parameters and arithmetic mean (CV %) was used to present the tₘₐₓ,
CL/F and Vz/F pharmacokinetic parameters.

| Dose | | | | | | | |
|---|---|---|---|---|---|---|---|
| Level | AUC₍₀₋ₜₗₐₛₜ₎ | AUC_{(0-∞)} | Cₘₐₓ | tₘₐₓ | t½ | CL/F | Vz/F |
| (μg) | (pg · h/mL) | (pg · h/mL) | (pg/mL) | (hour) | (h) | (L/h) | (L) |
| 10 | 10.8 (245.9) | — | 23.5 (58.2) | 0.250 (0.0) | — | — | — |
| | (n = 2) | | (n = 4) | (n = 4) | | | |
| 20 | 22.0 (112.5) | 92.0 | 33.2 (58.4) | 0.333 (38.7) | 0.546 | 217 | 171 |
| | (n = 4) | (n = 1) | (n = 6) | (n = 6) | (n = 1) | (n = 1) | (n = 1) |
| 40 | 75.5 (68.8) | 134 | 107.3 (37.0) | 0.339 (40.6) | 0.576 | 298 | 247 |
| | (n = 6) | (n = 1) | (n = 6) | (n = 6) | (n = 1) | (n = 1) | (n = 1) |
| 60 | 96.5 (84.8) | 139 (49.6) | 111.0 (52.4) | 0.293 (34.6) | 0.697 (11.6) | 466 (41.5) | 477 (48.7) |
| | (n = 6) | (n = 4) | (n = 6) | (n = 6) | (n = 4) | (n = 4) | (n = 4) |
| 80 | 264 (42.4) | 257 (19.7) | 233.6 (46.1) | 0.400 (34.2) | 0.779 (23.0) | 314 (19.4) | 361 (41.4) |
| | (n = 5) | (n = 2) | (n = 5) | (n = 5) | (n = 2) | (n = 2) | (n = 2) |

TABLE 4
Pharmacokinetic results of [Leu²⁷]cyclo(Glu²²-Lys²⁶)-hPTH-
(1-31)-NH₂ per dose level in plasma on Day 7. Geometric mean
(CV %) was used to present the AUC₍₀₋ₜₗₐₛₜ₎, AUC_{(0-∞)}, and
Cₘₐₓ pharmacokinetic parameters and arithmetic mean (CV %) was
used to present the tₘₐₓ, CL/F and Vz/F pharmacokinetic parameters.

| | AUC_{(0-τ)} | Cₘₐₓ | tₘₐₓ |
|---|---|---|---|
| Dose Level (μg) | (pg · h/mL) | (pg/mL) | (hour) |
| 10 | 20.6 (152.7) | 31.3 (58.6) | 0.250 (0.0) |
| | (n = 4) | (n = 5) | (n = 5) |
| 20 | 22.8 (134.7) | 39.1 (64.4) | 0.417 (72.7) |
| | (n = 6) | (n = 6) | (n = 6) |
| 40 | 107 (119.6) | 127.5 (36.4) | 0.250 (0.0) |
| | (n = 6) | (n = 6) | (n = 6) |
| 60 | 118 (59.8) | 132.7 (56.0) | 0.250 (0.0) |
| | (n = 6) | (n = 6) | (n = 6) |
| 80 | 286 (63.8) | 254.3 (74.6) | 0.261 (6.0) |
| | (n = 2) | (n = 2) | (n = 2) |

The mean t_{1/2} was consistent across dose groups, ranging from 0.546 hour (20 μg dose) to 0.779 hour (80 μg dose), indicating that the elimination of the peptide is independent of dose over the dose range studied.

Dose Proportionality 23

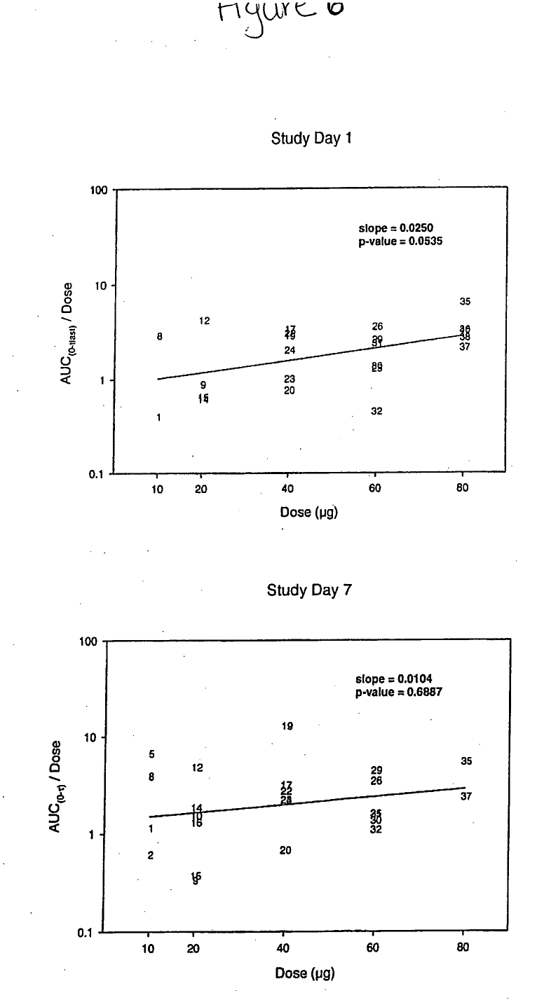

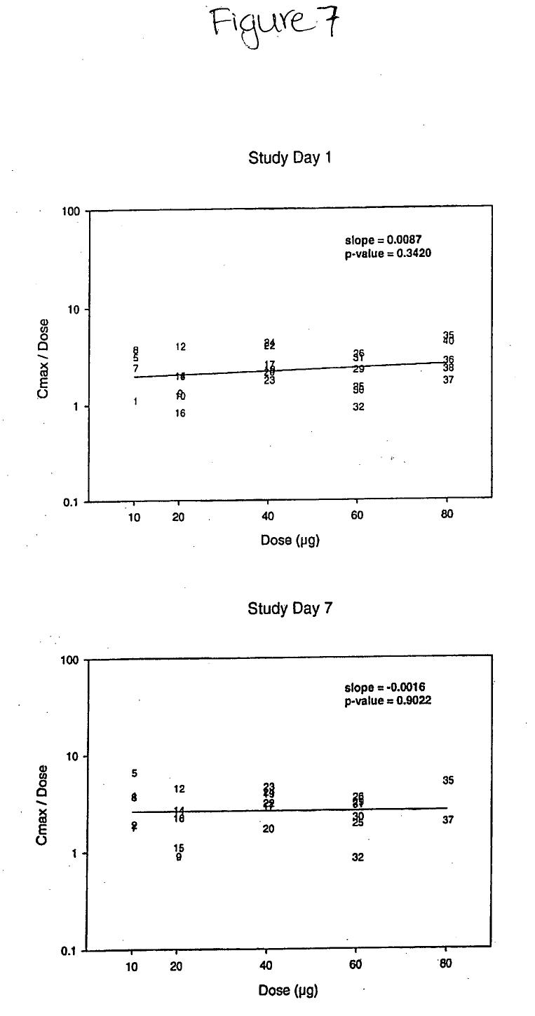

As shown in Table 5, AUC_{(0-∞)} (Day 1), AUC_{(0-τ)} (Day 7), Cₘₐₓ (Day 1) and Cₘₐₓ (Day 7) appear to be dose proportional with increasing dose from 10 μg to 80 μg (also see FIGS. 6 and 7).

TABLE 5
Dose proportionality (Days 1 and 7), dose linearity and accumulation
factor results of [Leu²⁷]cyclo(Glu²²-Lys²⁶)-hPTH-(1-31)-NH₂ per
dose level in plasma.

| | Dose Proportionality | | Dose Proportionality | | | Accumulation |
|---|---|---|---|---|---|---|
| | Day 1 | | Day 7 | | Dose Linearity | Factor |
| | AUC₍₀₋ₜₗₐₛₜ₎/ | Cₘₐₓ/ | AUC_{(0-τ)}/ | Cₘₐₓ/ | AUC_{(0-τ) Day 7/} | AUC_{(0-τ) Day 7/} |
| Dose Level (μg) | Dose | Dose | Dose | Dose | AUC_{(0-∞) Day 1} | AUC_{(0-tlast) Day 1} |
| 10 | 1.08 | 2.35 | 2.06 | 3.13 | — | 1.92 |
| | (n = 2) | (n = 4) | (n = 4) | (n = 5) | | |
| 20 | 1.10 | 1.66 | 1.14 | 1.95 | NC | 1.04 |
| | (n = 4) | (n = 6) | (n = 6) | (n = 6) | | |
| 40 | 1.89 | 2.68 | 2.68 | 3.19 | NC | 1.42 |
| | (n = 6) | (n = 6) | (n = 6) | (n = 6) | | |
| 60 | 1.61 | 1.85 | 1.96 | 2.21 | 0.85 | 1.22 |
| | (n = 6) | (n = 6) | (n = 6) | (n = 6) | | |
| 30 | 3.30 | 2.92 | 3.58 | 3.18 | 1.11 | 1.09 |
| | (n = 5) | (n = 5) | (n = 2) | (n = 2) | | |

### Pharmacokinetic Linearity and Accumulation Assessments:

Comparison between AUC values (AUC_{(0-τ)}Day 7/AUC_{(0-∞)} Day 1) indicated that the pharmacokinetic behavior of the peptide was linear after a once-daily subcutaneous administration for 7 days of 60 μg and 80 μg doses (see Table 5).

Overall, no observed accumulation was observed at the 20 μg, 60 μg and 80 μg dose levels.

While this invention has been particularly shown and described with references to embodiments thereof, it will be understood by those skilled in the art that various changes in form and details may be made therein without departing from the scope of the invention encompassed by the appended claims.

What is claimed is:

1. A method of treating a subject in need of hematopoietic stimulation, comprising the step of administering to said subject an effective amount of a peptide optionally in combination with an effective amount of a hematopoietic growth factor, wherein the peptide is:
(i) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1), where
Xaa=Lys, Leu, Ile, Nle or Met; or
(ii) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶,
Xaa=Leu, Ile, Nle or Met, and
Y=NH₂ or OH.

2. The method of claim 1, wherein an effective amount of said peptide is administered to said subject in combination with an effective amount of a hematopoietic growth factor.

3. The method of claim 1, wherein an effective amount of said peptide is administered to said subject in the absence of a hematopoietic growth factor.

4. The method of claim 1, wherein the peptide is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂, where Xaa=Lys, Leu, Ile, Nle, or Met.

5. The method of claim 4, wherein Xaa=Lys.

6. The method of claim 1, wherein the peptide is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y, cyclized in the form of a lactam between Glu²² and Lys²⁶,
Xaa=Leu, Ile, Nle or Met,
Y=NH₂ or OH.

7. The method of claim 6, wherein Xaa=Leu and Y=NH₂.

8. The method of claim 1, wherein the subject in need of hematopoietic stimulation is administered from 5 μg /day to 150 μg/day of the peptide.

9. The method of claim 1, wherein the subject in need of hematopoietic stimulation is administered from 30 μg/day to 100 μg/day of the peptide.

10. The method of claim 1, wherein the subject in need of hematopoietic stimulation is administered from 40 μg/day to 70 μg/day of the peptide.

11. The method of claim 1, wherein the subject in need of hematopoietic stimulation is administered from 55 μg/day to 65 μg/day of the peptide.

12. The method of claim 1, wherein the subject in need of hematopoietic stimulation is administered an effective amount of the peptide that results in the maximum plasma concentration of the peptide in the range of 10 pg/mL to 400 pg/mL.

13. The method of claim 1, wherein the subject in need of hematopoietic stimulation is administered an effective amount of the peptide that results in the maximum plasma concentration of the peptide in the range of 20 pg/mL to 300 pg/mL.

14. The method of claim 1, wherein the subject in need of hematopoietic stimulation is administered an effective amount of the peptide that results in the maximum plasma concentration of the peptide in the range of 50 pg/mL to 280 pg/mL.

15. The method of claim 1, wherein the subject in need of hematopoietic stimulation is administered an effective amount of the peptide that results in the maximum plasma concentration of the peptide in the range of 80 pg/mL to 250 pg/mL.

16. The method of claim 1, wherein the subject in need of hematopoietic stimulation is administered an effective amount of the peptide that results in the maximum plasma concentration of the peptide in the range of 100 pg/mL to 150 pg/mL.

17. The method of claim 1, wherein the subject in need of hematopoietic stimulation is administered an effective amount of the peptide that results in the value for area under the curve in a plasma concentration of the peptide versus time curve in the range of 5 μpg·h/mL-400 pg·h/mL.

18. The method of claim 1, wherein the subject in need of hematopoietic stimulation is administered an effective amount of the peptide that results in the value for area under the curve in a plasma concentration of the peptide versus time curve in the range of 10 pg·h/mL-350 pg·h/mL.

19. The method of claim 1, wherein the subject in need of hematopoietic stimulation is administered an effective amount of the peptide that results in the value for area under the curve in a plasma concentration of the peptide versus time curve in the range of 20 pg·h/mL-300 pg·h/mL.

20. The method of claim 1, wherein the subject in need of hematopoietic stimulation is administered an effective amount of the peptide that results in the value for area under the curve in a plasma concentration of the peptide versus time curve in the range of 50 μpg·h/mL-250 pg·h/mL.

21. The method of claim 1, wherein the subject in need of hematopoietic stimulation is administered an effective amount of the peptide that results in the value for area under the curve in a plasma concentration of the peptide versus time curve in the range of 70 pg·h/mL-200 μpg·h/mL.

22. The method of claim 1, wherein the subject in need of hematopoietic stimulation is administered an effective amount of the peptide that results in the value for area under the curve in a plasma concentration of the peptide versus time curve in the range of 90 pg·h/mL-150 μpg·h/mL.

23. The method of claim 1, wherein the subject in need of hematopoietic stimulation is administered an effective amount of the peptide that results in the value for area under the curve in a plasma concentration of the peptide versus time curve in the range of 95 μpg·h/mL-125 μpg·h/mL.

24. The method of claim 1, wherein said hematopoietic growth factor is a colony stimulating factor.

25. The method of claim 24, wherein said colony stimulating factor is selected from the group consisting of granulocyte colony-stimulating factor, granulocyte-macrophage colony-stimulating factor, macrophage colony-stimulating factor and multi-colony-stimulating factor.

26. The method of claim 24, wherein said colony stimulating factor is granulocyte colony-stimulating factor.

27. The method of claim 1, wherein said hematopoietic growth factor is a erythropoiesis regulator.

28. The method of claim 27, wherein said erythropoiesis regulator is erythropoietin.

29. The method of claim 1, wherein said hematopoietic growth factor is selected from the group consisting of thrombopoietin, Oncostatin M and interleukins.

30. The method of claim 1, wherein said subject is in need of hematopoietic stimulation as a consequence of a bone marrow transplantation.

31. The method of claim 30, wherein said bone marrow transplantation is an autologous bone marrow transplantation.

32. The method of claim 30, wherein said bone marrow transplantation is an alloegeneic bone marrow transplantation.

33. The method of claim 30, wherein said subject is a bone marrow donor.

34. The method of claim 30, wherein said subject is a bone marrow recipient.

35. The method of claim 1, wherein said subject is in need of hematopoietic stimulation as a consequence of having undergone chemotherapy.

36. The method of claim 1, wherein said subject is in need of hematopoietic stimulation as a consequence of having undergone radiation therapy.

37. The method of claim 1, wherein said subject is in need of hematopoietic stimulation as a consequence of having a hematological cancer.

38. The method of claim 37, wherein said cancer is selected from the group consisting of myeloma, lymphoma and leukemia.

39. The method of claim 37, wherein said cancer is acute myeloid leukemia.

40. The method of claim 1, wherein said subject is in need of hematopoietic stimulation as a consequence of having myelodysplastic syndrome.

41. The method of claim 1, wherein said subject is in need of hematopoietic stimulation as a consequence of having myelosuppression.

42. The method of claim 41, wherein said myelosuppression results in neutropenia.

43. The method of claim 1, wherein said subject is in need of hematopoietic stimulation as a consequence of having immunodeficiency.

44. The method of claim 1, wherein said subject is in need of hematopoietic stimulation as a consequence of a cord blood transplantation.

45. The method of claim 1, wherein said subject in need of hematopoietic stimulation is a human.

46. A method of neutrophil stimulation in a subject in need thereof, comprising the step of administering to said subject an effective amount of a peptide optionally in combination with an effective amount of a hematopoietic growth factor, wherein the peptide is:
(i) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-NH₂ (SEQ ID NO: 1), where
Xaa=Lys, Leu, Ile, Nle, or Met; or
(ii) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶,
Xaa=Leu, Ile, Nle or Met,
Y=NH₂ or OH.

47. The method of claim 46, wherein an effective amount of said peptide is administered to said subject in combination with an effective amount of a hematopoietic growth factor.

48. The method of claim 46, wherein an effective amount of said peptide is administered to said subject in the absence of a hematopoietic growth factor.

49. The method of claim 46, wherein the peptide is Ser-Val-Ser-Glu-lle-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂, where Xaa=Lys, Leu, Ile, Nle, or Met.

50. The method of 49, wherein Xaa=Lys.

51. The method of claim 46, wherein the peptide is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y, cyclized in the form of a lactam between Glu²² and Lys²⁶,
Xaa=Leu, Ile, Nle or Met,
Y=NH₂ or OH.

52. The method of claim 51, wherein Xaa=Leu and Y=NH₂.

53. The method of claim 46, wherein the subject in need of neutrophil stimulation is administered from 5 μg/day to 150 μg/day of the peptide.

54. The method of claim 46, wherein the subject in need of neutrophil stimulation is administered from 30 μg/day to 100 μg/day of the peptide.

55. The method of claim 46, wherein the subject in need of neutrophil stimulation is administered from 40 μg/day to 70 μg/day of the peptide.

56. The method of claim 46, wherein the subject in need of neutrophil stimulation is administered from 55 μg/day to 65 μg/day of the peptide.

57. The method of claim 46, wherein the subject in need of neutrophil stimulation is administered an effective amount of the peptide that results in the maximum plasma concentration of the peptide in the range of 10 pg/mL to 400 pg/mL.

58. The method of claim 46, wherein the subject in need of neutrophil stimulation is administered an effective amount of the peptide that results in the maximum plasma concentration of the peptide in the range of 20 pg/mL to 300 pg/mL.

59. The method of claim 46, wherein the subject in need of neutrophil stimulation is administered an effective amount of the peptide that results in the maximum plasma concentration of the peptide in the range of 50 pg/mL to 280 pg/mL.

60. The method of claim 46, wherein the subject in need of neutrophil stimulation is administered an effective amount of the peptide that results in the maximum plasma concentration of the peptide in the range of 80 pg/mL to 250 pg/mL.

61 The method of claim 46, wherein the subject in need of neutrophil stimulation is administered an effective amount of the peptide that results in the maximum plasma concentration of the peptide in the range of 100 pg/mL to 150 pg/mL.

62. The method of claim 46, wherein the subject in need of neutrophil stimulation is administered an effective amount of the peptide that results in the value for area under the curve in a plasma concentration of the peptide versus time curve in the range of 5 μpg·h/mL-400 μpg·h/mL.

63. The method of claim 46, wherein the subject in need of neutrophil stimulation is administered an effective amount of the peptide that results in the value for area under the curve in a plasma concentration of the peptide versus time curve in the range of 10 μpg·h/mL-350 μpg·h/mL.

64. The method of claim 46, wherein the subject in need of neutrophil stimulation is administered an effective amount of the peptide that results in the value for area under the curve in a plasma concentration of the peptide versus time curve in the range of 20 μpg·h/mL-300 μpg·h/mL.

65. The method of claim 46, wherein the subject in need of neutrophil stimulation is administered an effective amount of the peptide that results in the value for area under the curve in a plasma concentration of the peptide versus time curve in the range of 50 μpg·h/mL-250 μpg·h/mL.

66. The method of claim 46, wherein the subject in need of neutrophil stimulation is administered an effective amount of the peptide that results in the value for area under the curve in a plasma concentration of the peptide versus time curve in the range of 70 μpg·h/mL-200 μpg·h/mL.

67. The method of claim 46, wherein the subject in need of neutrophil stimulation is administered an effective amount of the peptide that results in the value for area under the curve in a plasma concentration of the peptide versus time curve in the range of 90 pg·h/mL-150 pg·h/mL.

68. The method of claim 46, wherein the subject in need of neutrophil stimulation is administered an effective amount of the peptide that results in the value for area under the curve in a plasma concentration of the peptide versus time curve in the range of 95 pg·h/mL-125 pg·h/mL.

69. The method of claim 46, wherein said hemotopoietic growth factor is a colony stimulating factor.

70. The method of claim 69, wherein said colony stimulating factor is selected from the group consisting of granulocyte colony-stimulating factor, granulocyte-macrophage colony-stimulating factor, macrophage colony-stimulating factor and multi-colony-stimulating factor.

71. The method of claim 69, wherein said colony stimulating factor is granulocyte colony-stimulating factor.

72. The method of claim 46, wherein said hemotopoietic growth factor is a erythropoiesis regulator.

73. The method of claim 72, wherein said erythropoiesis regulator is erythropoietin.

74. The method of claim 46, wherein said hemotopoietic growth factor is selected from the group consisting of thrombopoietin, Oncostatin M and interleukins.

75. The method of claim 46, wherein said subject is in need of neutrophil stimulation as a consequence of a bone marrow transplantation.

76. The method of claim 75, wherein said bone marrow transplantation is an autologous bone marrow transplantation.

77. The method of claim 75, wherein said bone marrow transplantation is an alloegeneic bone marrow transplantation.

78. The method of claim 75, wherein said subject is a bone marrow donor.

79. The method of claim 75, wherein said subject is a bone marrow recipient.

80. The method of claim 46, wherein said subject is in need of neutrophil stimulation as a consequence of having undergone chemotherapy.

81. The method of claim 46, wherein said subject is in need of neutrophil stimulation as a consequence of having undergone radiation therapy.

82. The method of claim 46, wherein said subject is in need of neutrophil stimulation as a consequence of having a hematological cancer.

83. The method of claim 82, wherein said cancer is selected from the group consisting of myeloma, lymphoma and leukemia.

84. The method of claim 82, wherein said cancer is acute myeloid leukemia.

85. The method of claim 46, wherein said subject is in need of neutrophil stimulation as a consequence of having myelodysplastic syndrome.

86. The method of claim 46, wherein said subject is in need of neutrophil stimulation as a consequence of having myelosuppression.

87. The method of claim 86, wherein said myelosuppression results in neutropenia.

88. The method of claim 46, wherein said subject is in need of neutrophil stimulation as a consequence of having immunodeficiency.

89. The method of claim 88, wherein said subject is in need of neutrophil stimulation as a consequence of a cord blood transplantation.

90. The method of claim 46, wherein said subject in need of neutrophil stimulation is a human.

91. A method of platelet stimulation in a subject in need thereof, comprising the step of administering to said subject an effective amount of a peptide optionally in combination with an effective amount of a hematopoietic growth factor, wherein the peptide is:
(i) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂ (SEQ ID NO: 1), where
Xaa=Lys, Leu, Ile, Nle, or Met; or
(ii) Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y (SEQ ID NO: 3), cyclized in the form of a lactam between Glu²² and Lys²⁶,
Xaa=Leu, Ile, Nle or Met, Y=NH₂ or OH.

92. The method of claim 91, wherein an effective amount of said peptide is administered to said subject in combination with an effective amount of a hematopoietic growth factor.

93. The method of claim 91, wherein an effective amount of said peptide is administered to said subject in the absence of a hematopoietic growth factor.

94. The method of claim 91, wherein the peptide is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-NH₂, where Xaa=Lys, Leu, le, Nle, or Met.

95. The method of 94, wherein Xaa=Lys.

96. The method of claim 91, wherein the peptide is Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Xaa-Leu-Gln-Asp-Val-Y, cyclized in the form of a lactam between Glu²² and Lys²⁶,
Xaa=Leu, Ile, Nle or Met,
Y=NH₂ or OH.

97. The method of claim 96, wherein Xaa=Leu and Y=NH₂.

98. The method of claim 91, wherein the subject in need of platelet stimulation is administered from 5 μg/day to 150 μg/day of the peptide.

99. The method of claim 91, wherein the subject in need of platelet stimulation is administered from 30 μg/day to 100 μg/day of the peptide.

100. The method of claim 91, wherein the subject in need of platelet stimulation is administered from 40 μg/day to 70 μg/day of the peptide.

101. The method of claim 91, wherein the subject in need of platelet stimulation is administered from 55 μg/day to 65 μg/day of the peptide.

102. The method of claim 91, wherein the subject in need of platelet stimulation is administered an effective amount of the peptide that results in the maximum plasma concentration of the peptide in the range of 10 pg/mL to 400 pg/mL.

103. The method of claim 91 wherein the subject in need of platelet stimulation is administered an effective amount of the peptide that results in the maximum plasma concentration of the peptide in the range of 20 pg/mL to 300 pg/mL.

104. The method of claim 91, wherein the subject in need of platelet stimulation is administered an effective amount of the peptide that results in the maximum plasma concentration of the peptide in the range of 50 pg/mL to 280 pg/mL.

105. The method of claim 91, wherein the subject in need of platelet stimulation is administered an effective amount of the peptide that results in the maximum plasma concentration of the peptide in the range of 80 pg/mL to 250 pg/mL.

106. The method of claim 91, wherein the subject in need of platelet stimulation is administered an effective amount of the peptide that results in the maximum plasma concentration of the peptide in the range of 100 pg/mL to 150 pg/mL.

107. The method of claim 91, wherein the subject in need of platelet stimulation is administered an effective amount of the peptide that results in the value for area under the curve in a plasma concentration of the peptide versus time curve in the range of 5 pg·h/mL-400 pg·h/mL.

108. The method of claim 91, wherein the subject in need of platelet stimulation is administered an effective amount of the peptide that results in the value for area under the curve in a plasma concentration of the peptide versus time curve in the range of 10 μpg·h/mL-350 μpg·h/mL.

109. The method of claim 91, wherein the subject in need of platelet stimulation is administered an effective amount of the peptide that results in the value for area under the curve in a plasma concentration of the peptide versus time curve in the range of 20 μpg·h/mL-300 μpg·h/mL.

110. The method of claim 91, wherein the subject in need of platelet stimulation is administered an effective amount of the peptide that results in the value for area under the curve in a plasma concentration of the peptide versus time curve in the range of 50 pg·h/mL-250 μpg·h/mL.

111. The method of claim 91, wherein the subject in need of platelet stimulation is administered an effective amount of the peptide that results in the value for area under the curve in a plasma concentration of the peptide versus time curve in the range of 70 μpg·h/mL-200 μpg·h/mL.

112. The method of claim 91, wherein the subject in need of platelet stimulation is administered an effective amount of the peptide that results in the value for area under the curve in a plasma concentration of the peptide versus time curve in the range of 90 pg·h/mL-150 μpg·h/mL.

113. The method of claim 91, wherein the subject in need of platelet stimulation is administered an effective amount of the peptide that results in the value for area under the curve in a plasma concentration of the peptide versus time curve in the range of 95 μpg·h/mL-125 μpg·h/mL.

114. The method of claim 91, wherein said hemotopoietic growth factor is a colony stimulating factor.

115. The method of claim 114, wherein said colony stimulating factor is selected from the group consisting of granulocyte colony-stimulating factor, granulocyte-macrophage colony-stimulating factor, macrophage colony-stimulating factor and multi-colony-stimulating factor.

116. The method of claim 114, wherein said colony stimulating factor is granulocyte colony-stimulating factor.

117. The method of claim 91, wherein said hemotopoietic growth factor is a erythropoiesis regulator.

118. The method of claim 117, wherein said erythropoiesis regulator is erythropoietin.

119. The method of claim 91, wherein said hemotopoietic growth factor is selected from the group consisting of thrombopoietin, Oncostatin M and interleukins.

120. The method of claim 91, wherein said subject is in need of platelet stimulation as a consequence of a bone marrow transplantation.

121. The method of claim 120, wherein said bone marrow transplantation is an autologous bone marrow transplantation.

122. The method of claim 120, wherein said bone marrow transplantation is an alloegeneic bone marrow transplantation.

123. The method of claim 120, wherein said subject is a bone marrow donor.

124. The method of claim 120, wherein said subject is a bone marrow recipient.

125. The method of claim 91, wherein said subject is in need of platelet stimulation as a consequence of having undergone chemotherapy.

126. The method of claim 91, wherein said subject is in need of platelet stimulation as a consequence of having undergone radiation therapy.

127. The method of claim 91, wherein said subject is in need of platelet stimulation as a consequence of having a hematological cancer.

128. The method of claim 127, wherein said cancer is selected from the group consisting of myeloma, lymphoma and leukemia.

129. The method of claim 127, wherein said cancer is acute myeloid leukemia.

130. The method of claim 91, wherein said subject is in need of platelet stimulation as a consequence of having myelodysplastic syndrome.

131. The method of claim 91, wherein said subject is in need of platelet stimulation as a consequence of having myelosuppression.

132. The method of claim 131, wherein said myelosuppression results in neutropenia.

133. The method of claim 91, wherein said subject is in need of platelet stimulation as a consequence of having immunodeficiency.

134. The method of claim 91, wherein said subject is in need of platelet stimulation as a consequence of a cord blood transplantation.

135. The method of claim 91, wherein said subject in need of platelet stimulation is a human.


SEQUENCE LISTING (4 sequences)

SEQ ID NO: 1 - protein, 31 aa, Artificial Sequence
  Chemically Synthesized
SVSEIQLMHNLGKHLNSMERVEWLRKXLQDV

SEQ ID NO: 2 - protein, 31 aa, Artificial Sequence
  Chemically Synthesized
SVSEIQLMHNLGKHLNSMERVEWLRKKLQDV

SEQ ID NO: 3 - protein, 31 aa, Artificial Sequence; identical to SEQ ID NO: 1
  Chemically Synthesized

SEQ ID NO: 4 - protein, 31 aa, Artificial Sequence
  Chemically Synthesized
SVSEIQLMHNLGKHLNSMERVEWLRKLLQDV